(19) United States
(12) Patent Application Publication
(10) Pub. No.: US 2010/0143878 A1
(43) Pub. Date: Jun. 10, 2010

(54) METHODS AND KITS FOR ISOLATING CELLS

(75) Inventors: Ryan J. Olson, Madison, WI (US); Rex M. Bitner, Cedarburg, WI (US); Allan M. Tereba, Madison, WI (US); Laura L. Bozek, Madison, WI (US)

Correspondence Address: MICHAEL BEST & FRIEDRICH LLP, 100 East Wisconsin Avenue, Suite 3300, Milwaukee, WI, 53202, US

(73) Assignee: PROMEGA CORPORATION, Madison, WI (US)

(21) Appl. No.: 12/444,075

(22) PCT Filed: Oct. 8, 2007
(86) PCT No.: PCT/US07/80686

§ 371 Date: Feb. 15, 2010

Related U.S. Application Data
(60) Provisional application No. 60/828,537, filed on Oct. 6, 2006.
(60) Provisional application No. 60/894,818, filed on Mar. 14, 2007.

(51) Int. Cl. A01N 1/02; C12N 1/08; C12N 1/00

(52) U.S. Cl. 435/2; 435/270; 435/267

(57) ABSTRACT

Disclosed are methods for differential extraction of a target component from a sample which is predominantly composed of other types of non-target cells that can be lysed using methods that do not lyse the target cells, so that the target material can be purified away from the lysed non-target material. One exemplary method is directed to isolating sperm cells from an aqueous sample and kits for performing same.

### CROSS-REFERENCE TO RELATED APPLICATIONS

This application claims priority to U.S. Provisional Application No. 60/828,537, filed Oct. 6, 2006 and to U.S. Provisional Application No. 60/894,818, filed Mar. 14, 2007.

### STATEMENT REGARDING FEDERALLY SPONSORED RESEARCH

Not applicable.

### INTRODUCTION

Differential extraction is useful in isolating target cell material from a sample that contains or is composed predominantly of other types of non-target cells. The non-target cells are preferentially lysed, so that target cells can be purified away from non-target material released from the lysed non-target cells. This is particularly useful when the target material is nucleic acid from a particular cell type to be used or detected in downstream nucleic acid amplifications because contaminating nucleic acids from non-target cells can obscure the target signal. Because the amount of nucleic acid template used in nucleic acid amplification must be limited, it is particularly helpful to reduce the contribution of non-target nucleic acids in the sample template. Contamination by non-target materials can also interfere with other detection methods, such as enzymatic detection or antibody-based detection of target materials. By lysing non-target cells during the purification process, background can be reduced, thereby enhancing the sensitivity and/or reliability of target material detection.

Examples of applications in which differential extraction can be useful include isolating sperm cells from forensics samples, isolating organisms that may be used as agents of biological warfare from diverse materials such as sewage, soil samples, air samples, or body fluids, and isolating organelles from cell samples.

Genetic material obtained from forensic samples can be used to identify perpetrators of sexual assaults or to exonerate innocent suspects. Purified DNA obtained from sperm cells isolated from forensic samples can be used in subsequent genetic identity testing. The genetic profile of sperm cell DNA can be compared to that of a known suspect or to databases containing genetic information about a large number of convicted felons.

In sexual assault cases, a forensic sample such as a vaginal or rectal swab, or clothing containing a semen stain, is obtained from the victim or crime scene for forensic analysis. If sperm cells are present in the sample, DNA from the sperm cells can be isolated and used in genetic identity testing. However, a vaginal swab obtained from a sexual assault victim typically contains relatively few sperm cells and large numbers of epithelial cells from the victim. As a consequence, unless the sperm cells are first separated from other cells in the sample, DNA purified from a forensic sample is susceptible to overwhelming contamination with epithelial cell DNA. Such contamination interferes with the ability to establish a match between the genetic profile of DNA from the sample and that of the suspect or a member of the database. It is therefore desirable to isolate sperm cells from other cells in a forensic sample prior to DNA isolation and analysis.

Techniques currently used to isolate sperm cells from other cells in forensic samples are time consuming and labor intensive, and there is currently a backlog of unprocessed samples. Because of this backlog, some jurisdictions have a policy against processing samples unless a suspect has been identified. Consequently, many unprocessed samples are ultimately discarded, and genetic information contained in the sample is never compared with or entered into the national database, which reduces the ability of law enforcement to identify and apprehend repeat sex offenders.

Sperm cells are typically isolated from forensic samples containing epithelial cells by selectively lysing the epithelial cells by treatment with Proteinase K and a detergent under nonreducing conditions (Gill et al. Nature 318:577-579, 1985). Following epithelial cell lysis, intact sperm cells are pelleted by centrifugation and the supernatant, which contains DNA from lysed epithelial cells, is removed. In order to minimize contamination by soluble epithelial cell DNA, the sperm pellet is subjected to repeated washing with an aqueous buffer in an attempt to remove soluble epithelial cell DNA. This process frequently results in the loss of sperm cells and is very labor intensive.

Sperm cells have been isolated from samples containing both sperm and epithelial cells by selectively binding the sperm cells to sperm cell specific polyclonal or monoclonal antibodies attached to a solid support (e.g., paramagnetic particles). After binding the cells to the immobilized antibodies, the support is washed to remove unbound cells. This method requires a large amount of antibodies and is, therefore, relatively expensive. Furthermore, the binding process is inefficient and sperm cells typically are lost during the wash steps, resulting in reduced yield and reduced sensitivity. Because sperm cells undergo structural changes in the relatively low pH of the vagina, many sperm cell-specific antibodies do not bind to sperm cells from all semen-containing samples. In addition, antibodies may not bind efficiently because of variations or mutations in sperm cell surface antigens in certain individuals, resulting in poor sperm cell yields.

Sperm cells may be separated from epithelial cells on the basis of differences in cell size by filtering the sample through size selective membranes. This method is problematic because sperm cells tend to become trapped among the epithelial cells, mucus and cell debris, the sperm cells form clumps that are too large to pass through the membrane, and the membrane tends to clog, which ultimately may result in low yields of sperm cells. In addition, DNA from lysed epithelial cells pass through the membrane with the sperm and contaminate the sperm.

In another method, sperm cells are isolated by first selectively lysing epithelial cells and filtering the lysate to effect separation of the soluble epithelial cell DNA and intact sperm cells. However, the method suffers from disadvantages, including clogging of the membrane, which results in contamination of the sperm cells with epithelial cell DNA.

In the field of reproductive medicine, sperm cells have been isolated from fresh semen using a cell sorter, which although effective, is not practical in the forensic context because it is costly, time consuming, and does not address how to effectively recover sperm cells and epithelial cells from a forensic sample (e.g., a swab or clothing).

Microdissection of sperm from a sample on a slide is another approach that obtains sperm free of contaminating epithelial cell DNA. While effective, this approach is not conducive to automation and is prone to selective bias in samples containing sperm from more than one contributor, a common occurrence in rape samples.

Thus, there is a need in the art for simplified methods of separating sperm cells from epithelial cells in forensic samples that can be used in automated processing.

### BRIEF SUMMARY OF THE INVENTION

In one aspect, the invention includes a method of isolating first cells from a sample comprising first cells and second cells by treating the sample to form an aqueous lysate conditions that preferentially lyse the second cells without lysing the first cells, applying a force to the sample effective to form a pellet comprising the first cells, and forming a pellet immobilizing cap between the pellet and the aqueous lysate.

In another aspect, the methods allow target organelles to be separated from an aqueous lysate sample by pelleting the intact target organelles and forming a pellet immobilizing cap between the pellet and the aqueous lysate.

### DETAILED DESCRIPTION OF THE INVENTION

Disclosed are methods suitable for isolating sperm cells from samples containing non-sperm cells, e.g., epithelial cells, by selectively lysing the non-sperm cells and then separating the intact sperm cells from lysed non-sperm cells using a pellet immobilizing cap. In addition to allowing isolation of sperm cells, these methods were found to be useful in isolating other cell types from samples comprising more than one cell type.

These methods are particularly useful in purifying a target cell from a sample including or composed predominantly of non-target cells that can be preferentially lysed by selecting conditions that lyse the non-target cells while the target cells are not lysed, or are lysed to a lesser extent than the non-target cells. This facilitates isolation of the target cells from lysed non-target cells. The isolated target cells may then be used to isolate, for example, target cell nucleic acid which can serve as a template in subsequent nucleic acid amplifications. Removal of non-target cell material reduces the problem of contaminating nucleic acids from non-target cells contributing background nucleic acid that can overwhelm or obscure the target signal.

The methods of the invention are applicable to any application in which one wishes to isolate a material from a cell in a starting material containing more than one type of cell that are differentially susceptible to lysis under certain conditions. The methods may be used, for example, to purify microorganisms from a wide range of potentially biologically complex materials such as sewage, soil samples, air samples, or body fluids. Microorganisms such as bacteria, viruses, yeast, and fungi, including, for example, naturally occurring pathogens, agents of biological warfare, toxic mold, and the like, can be purified using the methods of the invention. Additionally, the methods can be used to purify intact organelles such as nuclei from cell samples.

Forensic samples obtained from victims of sexual assault typically contain a large number of epithelial cells and, if present, relatively few sperm cells. In order to obtain DNA from the sperm cells for subsequent use in genetic identity testing, it is necessary to isolate the sperm cells from aqueous soluble material, especially DNA, that may interfere with or complicate the interpretation of results. For example, DNA from lysed epithelial cells is soluble in aqueous solutions.

Described herein below are methods in which the sperm cells are separated from soluble aqueous material in a sample by applying a force to the sample, typically by centrifugation, for a period of time sufficient to form a sperm pellet, and delivering a pellet immobilizing cap material to the sample to form a barrier between the sperm pellet and the aqueous material. The aqueous soluble material (e.g., DNA) remains in the aqueous phase and is physically separated from the pelleted sperm cells by the pellet immobilizing cap. The pellet immobilizing cap minimizes disruption of the sperm cell pellet that can occur, for example, when liquids are added to or removed from the container. The use of a pellet immobilizing cap according to the present invention facilitates removal of contaminating material (e.g., epithelial cell DNA), while minimizing loss of the sperm cell pellet. Loose sperm cell pellets formed by relatively low centrifugal forces are particularly susceptible to disruption. Therefore, the methods of the invention are particularly suitable in high throughput systems employing multiwell plates, which are adapted for use in plate rotors capable of applying only relatively low centrifugal forces. The methods are also useful in separating tightly packed sperm cell pellets formed under relatively high centrifugal forces from contaminating material.

As used herein, “sperm cells” may include an intact sperm cell or essentially intact sperm cell, and a sperm cell that has lost its flagellum or “tail”, and includes immature sperm cell precursors such as spermatids. Although a sperm cell may have been exposed to environmental conditions, mechanical sheering, or chemical treatment (e.g., exposure to Proteinase K or other agents) that altered the cell, such cells are within the scope of the invention, especially those cells that retain their nuclei.

Forensic samples from sexual assault victims are typically collected on a solid support, such as a swab or cloth (e.g., a cutting from clothing containing a semen stain). The swab or cloth may be transferred to a container such as a test tube or other suitable container and contacted with an aqueous solution such as a lysis buffer, for example, that preferentially lyses non-sperm cells. Following transfer of the material from the solid support to the aqueous solution, the container is centrifuged to effect sedimentation or pelleting of the sperm cells within the aqueous material. Optionally, recovery of the aqueous buffer and sperm cells may be facilitated by transferring the solid support and aqueous buffer to a second container equipped with a mechanical barrier that effectively prevents passage of the solid support while allowing the liquid sample to pass. Recovery of the aqueous sample from the solid support and pelleting of the sperm cells may be accomplished in a single centrifugation.

The lysis buffer used to selectively lyse the epithelial cells suitably comprises Proteinase K, and Sarkosyl or SDS. Optionally, the lysis buffer may comprise any suitable water soluble dye (e.g., FD & C Yellow) to enhance visualization of the aqueous phase. The material is treated with a suitable amount of a protease, such as Proteinase K, under conditions that allow lysis of the epithelial cells, but do not promote lysis of the sperm cells. Sperm cells are relatively resistant to proteases because the exposed proteins contain a relatively large number of disulfide bonds. Therefore, reducing conditions are not suitable for differential lysis because the presence of reducing agents would disrupt the disulfide bonds and increase lysis of sperm cells treated with proteases. It is also envisioned that digitonin or non-ionic detergents such as Tergitol may be used to selectively permeabilize or lyse different types of cells. It is expected that because of their structures, epithelial cells are more sensitive to permeabilization or lysis by digitonin than are sperm cells. Similarly, it is expected that cells having different cholesterol content will exhibit differential susceptibility to permeabilization by digitonin.

After treatment with the Proteinase K and detergent, the lysate containing the lysed epithelial cells and intact sperm cells is centrifuged to pellet the sperm cells. A pellet immobilizing cap material is then transferred to the container to form a pellet immobilizing cap between the pelleted sperm cells and the aqueous material. Preferably, the pellet immobilizing cap layer is transferred near the lower portion of the aqueous material, just above the sperm cell pellet. The aqueous material is then removed, while the pellet immobilizing cap remains in place over the pellet.

In the Examples below, MagneSil® paramagnetic particles (Promega Corp.) and DNA IQ® resin (cat#A8252, Promega Corp., Madison, Wis.) were used as a pellet immobilizing cap material to form a pellet immobilizing cap between the cell pellet and non-target material (e.g., aqueous material or non-target cell lysate). The pellet immobilizing cap was held in place between the pellet and non-target material by placing a magnet under the cell pellet during removal of the aqueous material to prevent mixing and disruption of the pellet. Once the aqueous material was removed and the cell pellet washed, the cells in the cell pellet were lysed under conditions that allow the DNA from the cells to bind to the particles.

It is envisioned that other suitable paramagnetic materials may be used to form a pellet immobilizing cap over the cell pellet to prevent disruption of the sperm cell pellet during removal of the aqueous material, including, but not limited to those described in U.S. Pat. No. 6,673,631, titled “Simultaneous Isolation and Quantitation of DNA,” to Tereba et al and U.S. Pat. No. 6,027,945, titled “Methods of Isolating Biological Target Materials Using Silica Magnetic Particles,” to Smith et al. In a preferred embodiment of the present invention, the total volume of magnetic or paramagnetic pellet immobilizing cap material will be about 40 ul or less. In a more preferred embodiment of the present invention, the total volume of magnetic or paramagnetic pellet immobilizing cap material per sample will be about 10 ul or less. In a most preferred embodiment of the present invention, the total volume of magnetic or paramagnetic pellet immobilizing cap material per sample will be about 2 ul or less.

The magnetic particles comprise ferrous material. It is specifically envisioned that other magnetic or paramagnetic materials could be employed, such as nickel or cobalt containing materials. The magnetic particles may be coated with material comprising silica or cellulosic compounds (e.g., cellulose, dextran, or agarose) such as those described in U.S. Pat. No. 6,855,499.

A preferred embodiment of the present invention utilizes aggregate magnetic or paramagnetic particles wherein each particle comprises two or more magnetic or paramagnetic cores that are covered by a silaceous oxide coating. A preferred size for such aggregate magnetic particles is 1-15 micron in the smallest dimension of the particle. A preferred particle BET surface area for these aggregate magnetic particles is from about 1 m²/gm to about 50 m²/gm as determined by nitrogen absorption.

Another suitable material for use as a pellet immobilizing cap is at least one magnetic mat comprising woven or unwoven fiber containing magnetic material, such as a moldable magnetic material commercially available from 3M. In operation, it would be suitably sized to form a layer between the pellet and the aqueous phase. The mat could be a solid surface or contain openings of sufficient size to allow passage of liquid but not the pelleted material. For example, the magnetic mat could comprise netting having an opening smaller than the smallest dimension of the target material (e.g., sperm). Use of multiple mats may provide additional assurance that the pellet is covered during the processing steps. Magnetic mats can be plastic moldable magnets. The plastic magnets are a low cost magnet that can be molded, stamped, or machined into a particular shape, are light weight, and have high magnetic strength. An example of such plastic magnets is Magnet Material B-1060 manufactured by 3M Electronics Products Division of St. Paul, Minn. Permanent magnets in the form of flexible ferrite sheets or strips available from Dexter Magnetic Technologies (Elk Grove Village, Ill.) could also be used to form a woven or unwoven mat.

Suitable materials for use as a pellet immobilizing cap include materials having a density greater than that of the aqueous material, i.e., greater than about 1.0 g/cm³. Preferably, the pellet immobilizing cap material is one that does not bind to the soluble aqueous material under the conditions used to effect separation between the sperm cell pellet and the aqueous material. Additionally, the material is preferably capable of displacing the aqueous material to form a pellet immobilizing cap over the sperm cell pellet while at the same time substantially excluding the aqueous material. Suitably, the pellet immobilizing cap material is configured to resist mixing as, for example, in response to the addition or removal of liquids to the system. In a preferred method of using the present invention, the pellet immobilizing cap is capable of being held in place by application of an external magnetic field or physically by a change in state of the pellet immobilizing cap, such as a change in temperature or pressure. While particle size is not critical, it is expected that relatively small particles with trap less aqueous solution and therefore will require less washing.

Examples of suitable pellet immobilizing cap materials include a temperature-responsive material. The temperature-responsive material is in a solid or semi-solid state at temperatures above about 32° C. and liquid form below about 32° C. Accordingly, the temperature-responsive material will be in liquid state at room temperature. The temperature responsive material is reversibly convertible from liquid to solid (or semi-solid state) and vice verse, so the pellet immobilizing cap is created by sufficiently heating the sample to convert the temperature-responsive material to a solid phase.

An exemplary temperature-responsive material is N-Isopropylacrylamide (NIPA) (also known as N-(n-Propyl)acrylamide; Registry No. 2210-25-5). Other suitable temperature responsive materials include poly(N,N-dimethylacrylamide), poly(ethylacrylamide), poly(N-ethylmethacrylamide), poly[N-(3 ethoxypropyl)acrylamide]; poly[N-(2-hydroxypropyl)methacrylamide]; poly(N-vinylisobutyramide), poly(N-vinylacetamide), a copolymer of methoxy poly(ethylene glycol) and polypropylene fumarate), a vinyl ether of ethylene glycol, hydroxypropylcellulose, ethyl hydroxyethyl cellulose, methyl cellulose, poly(vinyl methyl ether), butyl vinyl ether, polyglycidol, acryloyl-L-proline methyl ester, a vinyl pyrrolidone and vinyl acetate copolymer, a copolymer of N-acryloyl-N′-alkyl piperazine and methyl methacrylate, poly(methyl 2-propionamidoacrylate), poly(acrylic acid), poly(acrylamide-co-butyl methacrylate), poly(organophosphazenes), poly(2-ethyl-2-oxazoline), gelatin, poly(N-vinylcaprolactam), elastin, elastin mimetic polypeptide, 2,4,6 trimethylpyridine and poly(N-vinylpyrrolidone).

A non-aqueous liquid having a density greater than that of the aqueous material may optionally be added following the formation of the pellet immobilizing cap over the sperm cell pellet and prior to removal of the aqueous material to facilitate removal of the aqueous material. Non-aqueous liquids suitable for use in conjunction with the methods of the invention include those described in U.S. application Ser. No. 10/939,105, which is incorporated by reference herein. The density of the non-aqueous liquid is preferably intermediate between that of the aqueous material and than that of the pellet immobilizing cap material. However, if the pellet immobilizing cap material is maintained in place by another force material, for example, a magnetic force applied to a paramagnetic pellet immobilizing cap, a non-aqueous liquid having a density greater than that of the pellet immobilizing cap material may suitably be used. After adding the non-aqueous liquid, the aqueous and non-aqueous phases are separated optionally facilitated by centrifugation, and the aqueous phase is removed.

It is well within the ability of one skilled in the art to evaluate the suitability of non-aqueous liquids to facilitate removal of the aqueous material according to the method of the invention. The non-aqueous liquid is suitably non-chaotropic so as to prevent undesired lysis of the pelleted cells. The non-aqueous liquid is preferably one that has a relatively low solubility in water. Using a non-aqueous liquid having low solubility in water will typically afford better phase separation and reduced contamination of the sperm cell pellet with water soluble materials, such as epithelial cell DNA.

As described in detail in U.S. application Ser. No. 10/939,105, suitable non-aqueous liquids include, but are not limited to, diethyl glutarate (“DEG”), dimethyl glutarate (“DMG”), and 1-chloro-2-methyl-2-propanol. The density of the non-aqueous liquid may be adjusted by using two or more non-aqueous liquids having different densities in combination at ratios effective to obtain the desired density. When two or more non-aqueous liquids are used, the liquids are suitably substantially miscible with each other so as to form a liquid mixture of substantially uniform density. Alternatively, the extraction of DNA from the pellet may be performed using standard purification methods.

In addition, it is specifically envisioned that chloroform could be used in conjunction with DMG, DEG or 1-chloro-2-methyl-2-propanol to isolate sperm cells by selecting appropriate ratios to provide a mixed non-aqueous liquid having a suitable density.

Epithelial cell DNA from lysed epithelial cells, which is soluble in water, is found in the aqueous layer, and is separated by removing the aqueous material by any suitable means, including, for example, by pipetting. The epithelial cell DNA present in the aqueous phase may optionally be used as a control in genetic identity testing to confirm the source of the sample. Following recovery of the aqueous phase, a small amount of aqueous solution may remain at or near the surface of the barrier and on the container wall. Optionally, to enhance removal of the aqueous solution and epithelial cell DNA without disrupting the sperm cell pellet, the residual aqueous solution may be removed by washing with water or a suitable aqueous solution. Optionally, the pellet immobilizing cap material and sperm cells may be pelleted, and the aqueous wash removed. Optionally, additional pellet immobilizing cap material may be added to form an additional pellet immobilizing cap over the previously pelleted and capped sperm cells prior to removing the aqueous wash material. The additional pellet immobilizing cap material may be the same as or different than the pellet immobilizing cap material added during the initial purification.

As described in the Examples, the aqueous material may be removed to leave a sperm cell pellet, and the sperm cells may be lysed by contacting the cells with an aqueous solution comprising a detergent and/or chaotrope and may also contain DTT, followed by extraction of the DNA. Alternatively, the extraction of DNA from the pellet may be performed using standard purification methods.

For example, following removal of the aqueous phase, a lysis buffer containing a chaotropic agent and a reducing agent may be added to the non-aqueous liquid and vortexed to form a substantially homogenous mixture. As shown in the Examples, a lysis buffer containing guanidine thiocynate (GTC) and dithiothreitol (DTT) was combined with the non-aqueous phase, magnetic silica resin, and sperm cell pellet to effect lysis of the sperm cells and binding of DNA released from the cells upon lysis to the magnetic silica resin.

Alternatively, both the aqueous and non-aqueous layers may be removed to leave a sperm cell pellet, and the sperm cells present may be lysed by contacting the cells with an aqueous solution comprising a detergent, such as sodium dodecyl sulfate (SDS), and a reducing agent, such as DTT. Lysis may be followed by isolating DNA by extraction with phenol:chloroform or using any suitable DNA purification method, e.g., standard DNA purification methods known in the art.

As described in the Examples, the method of the invention was found to be effective in isolating sperm cells from epithelial cells contained within forensic samples such as vaginal or cervical swabs. It is envisioned that the method will be useful in isolating sperm cells from other sources, including other solid supports containing semen, such as cloth. It is reasonably expected that the method of the invention will be suitable for use with samples containing sperm cells and contaminating red or white blood cells or DNA derived from other nucleated non-sperm cells.

In addition to its suitability for use in isolating sperm cells, the method of the invention is expected to have general applicability in separating cells on the basis of differential sensitivities to various lysis conditions. For example, the method of the invention can be adapted to separate certain gram positive bacterial cells from gram negative cells following differential lysis of the gram negative cells by treatment with lysozyme, to which gram negative cells are particularly susceptible.

Additionally, the method is useful in isolating subcellular organelle material soluble in an aqueous solvent following selective lysis, for example, selective lysis of the cell membrane or cell wall. As described in the Examples below, the method has been found to be useful in isolating nuclei from other cellular substituents following cell lysis of whole blood. The method is useful in separating pelleted target material from cellular substituents in solution, and is also useful in separating target cellular substituents from non-target pelleted material.

Optionally, cell nuclei may be isolated using the methods of the invention by selectively lysing the cell membrane of a cell using conditions under which lysis of the nuclear membrane is minimized. Example 6 describes, prophetically, using the methods of the invention to isolate RNA from HEK cells by treating the cells with a lysis buffer that includes digitonin and RNasin. Under these conditions, it is expected that the cellular membrane will be at least partially lysed to allow release of cytoplasmic RNA from the cell, and that the cell nucleus will remain sufficiently intact to allow it to be pelleted by centrifugation, while the RNA remains in the lysate. Selective lysis or permeabilization using digitonin is described in greater detail in U.S. Provisional Application Ser. No. 60/894,810, filed Mar. 14, 2007, which is incorporated by reference in its entirety. A pellet immobilizing cap is then formed between the pellet and the lysate comprising the RNA.

Example 6 describes, prophetically, using a lysis buffer comprising 40 mg/ml digitonin. However, it is envisioned that digitonin may be used in any suitable concentration, provided that the concentration is effective in lysing the cell membrane, without causing substantial lysis or permeabilization of the nucleus. Substantial lysis of the nucleus is that which would interfere with the ability to pellet nuclei. Preferably, at least 5%, 10%, 25%, 50%, 75% or 99% of the nuclei can be removed by centrifugation. For example, it is envisioned that digitonin in concentrations of 0.5, 1, 2, 5, 10, 15, 20, 25, 30, 35, 40, 45, or 50 mg/ml would be suitable. Example 6 also describes, prophetically, including in the lysis buffer RNasin Plus at a concentration of 400 U/ml. It is envisioned that RNasin may be included in an amount effective to inhibit any RNases that may be present to an extent sufficient to allow isolation of at least some RNA. Preferably, RNasin is present in a concentration of at least 0.1 units/ul of lysate, 0.2 units/ul of lysate, 0.4 units/ul of lysate, 1.0 units/ul of lysate, 2 units/ul of lysate, 4 units/ul of lysate, 6 units/ul of lysate, 8 units/ul of lysate, or 12 units/ul of lysate. As one of skill in the art may appreciate, any suitable RNasin may be used.

Example 7 describes, prophetically, selective lysis of epithelial cells in a sample comprising both epithelial cells and sperm cells. Using a lysis buffer comprising digitonin or a nonionic detergent such as Tergitol, or a mixture thereof, is expected to reduce the amount of Proteinase K needed to lyse the epithelial cells, or to eliminate the need for Proteinase K altogether. Reduction of Pproteinase K may result in reduced degradation or lysis of sperm cells and improved yield of sperm cells and sperm cell nucleic acids.

Example 7 describes, prophetically, using a lysis buffer comprising 40 mg/ml digitonin. However, it is envisioned that digitonin may be used in any suitable concentration, provided that the concentration is effective in lysing the epithelial cells, without causing substantial lysis of the sperm cells. For example, it is envisioned that digitonin in concentrations of 0.5, 1, 2, 5, 10, 15, 20, 25, 30, 35, 40, 45, or 50 mg/ml would be suitable. Substantial lysis of the sperm cells is that which would interfere with the ability to pellet sperm cells. Preferably, at least 5%, 10%, 25%, 50%, 75% or 99% of the sperm cells can be removed by centrifugation.

Suitably, materials used in the methods of the invention may be provided as a kit. A kit suitably comprises a pellet immobilizing cap material, for example, paramagnetic particles, aggregate silica magnetic or paramagnetic particles or silica magnetic particles, such as MagneSil® particles. A kit of the present invention may also comprise one or more other components useful in isolating particular cells, subcellular organelles, or cellular components from particular cells. For example, the kits may contain a buffer or reagent used in the differential lysis of contaminating cells, for example, Proteinase K, lysozyme, or a detergent. Optionally, the kit may comprise components used in isolating DNA from the cell of interest, including, for example, a chaotropic agent. The kit may optionally contain a non-aqueous liquid of a suitable density for facilitating removal of aqueous materials.

The following non-limiting examples are intended to be purely illustrative.

### Example 1

### Pellet Immobilizing Cap Method Vs. Standard Method of Successive Spins and Washes

Samples used in this Example were dried sample swabs prepared from vaginal swabs to which were added semen diluted with nanopure water to yield the equivalent of 0.2 μl semen (˜32,000 sperm) per swab. The cotton substrate from each swab was divided in half to form two samples. 400 μl Digestion Buffer (Promega) with 108 μg Proteinase K (Promega catalog number V3022) was added to each sample within a 1.5 ml microcentrifuge tube. Samples were vortexed for 30 seconds and incubated at 56° C. for 1 hour. Following incubation, the cotton substrate portions of the swabs were removed from the solution with tweezers and transferred to spin baskets seated in fresh 1.5 ml microcentrifuge tubes. The remaining liquid digests were transferred by pipette to the spin baskets containing the corresponding cotton substrate. Samples were spun in a swinging-bucket rotor (All from Beckman: Allegra 6R centrifuge; GH-3.8 rotor; Microplus Carrier; Biomek2000 24-position tube rack with 1.5 ml microcentrifuge tube adapters) at 1,400×g (3,000 rpm) for 10 minutes. The spin baskets, which contained the cotton substrates, were removed from sample tubes and discarded. The samples were split into two treatment groups: Group 1 (pellet immobilizing cap) and Group 2 (Spin/Wash) for obtaining sperm fractions.

Group 1: 7 μl DNA IQ™ Resin (cat#A8252, Promega Corp.) was added to each spun sample tube. The tube was placed upon a flat magnet such that the magnetized DNA IQ™ Resin localized to the bottom portion of the tube where a cell pellet was seen or expected to be. 100 μl Differex™ Separation Solution (cat#A8512) was then added to each sample tube near the bottom of the aqueous digest but above the magnetized Resin. The aqueous digest fraction and the Separation Solution separated into an upper aqueous layer and a lower non-aqueous layer. The upper aqueous layer was removed. Tube walls and top surface of non-aqueous layer were washed with two serial 500 μl nanopure water washes. Following final wash removal, 200 μl DNA IQ™ Lysis Buffer (cat#A8262) with 60 mM DTT was added to each sample, and the samples were vortexed briefly. DNA isolation was performed using the DNA IQ™ nucleic acid isolation system (cat#DC6700). The DNA was eluted in a 40 μl volume.

Group 2: Following the centrifugation, a majority of digest supernatant was removed, leaving 15 to 20 μl of supernatant over the cell pellets. A 500 μl aliquot of nanopure water was added to each sample, and the samples were spun in a centrifuge for an additional 10 minutes. The supernatant was removed to leave 15 to 20 μl. The wash process was repeated twice for a total of three water washes. Following final wash removal, 100 μl DNA IQ Lysis Buffer with 60 mM DTT was added to each sample. 70 DNA IQ Resin was added to each sample. Samples were vortexed briefly and used as starting material for DNA IQ nucleic acid isolation. DNA isolations were eluted in 40 μl volume.

Sperm fraction samples from Groups 1 and 2 were quantified using Quantifiler Y assay system (Applied Biosystems, San Carlos Calif., catalog number 4343906). A THO1 single-locus STR amplification (Promega Corp, cat#DC6561) was performed for each sperm fraction sample, using 0.5 to 1.25 ng template. Capillary electrophoresis was performed (3100 Genetic Analyzer; Applied Biosystems) for male fraction sample by combining 1 μl of the THO1 amplification product with 9 μl Hi-Dye Formamide and 1 μl ILS600. Injection Parameters for Data resultant from the capillary electrophoresis was analyzed using Genescan software (Applied Biosystems).

At the THO1 locus, both female and male contributors were heterozygous. The female contributor displayed a genotype of (9.3, 9) and the male contributor displayed a genotype of (9, 6). Both contributors shared the 9-repeat allele at the THO1 locus. The unshared THO1 alleles between the two contributors were 9.3 repeats (female-specific) and 6 repeats (male-specific). The electropherogram peaks for the 9.3 and 6 base pair-repeat amplification products displayed in the fluorescein channel at approximately 175 and 161 base pairs, respectively.

Examination of the fluorescent strengths of these peaks, manifested as peak heights from the electropherograms, was used to determine relative representation of the two contributors in the male fraction samples. The results obtained for the two unshared allele peaks (female- and male-specific) are presented in Table 1 below.

TABLE 1

| | 175 bp (female-specific) | 161 bp (male-specific) |
|---|---|---|
| | peak height in Relative | peak height in Relative |
| Sample | Fluorescence Units | Fluorescence Units |
| Treatment | 329 | 739 |
| Group 1 | | |
| Replicate 1 | | |
| Treatment | 0 | 370 |
| Group 1 | | |
| Replicate 2 | | |
| Treatment | 0 | 1069 |
| Group 1 | | |
| Replicate 3 | | |
| Treatment | 0 | 184 |
| Group 2 | | |
| Replicate 1 | | |
| Treatment | 0 | 1205 |
| Group 2 | | |
| Replicate 2 | | |
| Treatment | 0 | 262 |
| Group 2 | | |
| Replicate 3 | | |

This data shows that the pellet immobilizing cap (Group 1) performs comparably to the traditional differential extraction method of serial epithelial fraction dilutions, spins and removals (Group 2) in separating sperm from the majority female epithelial cells into a male fraction that is predominantly free from female epithelial DNA carryover.

### Example 2

### Magnetic Immobilization of a Sperm Pellet Using DNA IQ™ Resin, with and without a Non-Aqueous Liquid, as a Pellet Immobilizing Cap

Samples used in this Example were dried sample swabs prepared from vaginal swabs to which semen diluted with nanopure water to yield the equivalent of 2 μl semen (˜200,000 sperm) per swab. The cotton substrate from each swab was divided in half to form two samples. 400 μl Digestion Buffer with 310 μg Proteinase K was added to each sample in 1.5 ml microcentrifuge tubes. Samples were vortexed for 30 seconds and incubated at 56° C. for 90 min. Following incubation, cotton substrates were removed from the solution with tweezers and transferred to spin baskets seated in fresh 1.5 ml microcentrifuge tubes. The remaining liquid digests were transferred by pipette to the spin baskets containing the corresponding cotton substrate. Samples were capped and spun in a swinging-bucket rotor at 1,400×g (3,000 rpm) for 10 minutes. The spin baskets, which contained the cotton substrates, were removed from sample tubes and discarded. Samples were placed into a MagneSphere Technology Magnetic Separation Stand (Promega catalog#Z5332) modified so that the magnetic field was concentrated at the cell pellet on the bottom of the tube. 7 μl DNA IQ™ Resin was then added to each sample tube. Samples were split into two treatment groups: Group 1 (DNA IQ™ Resin sperm pellet immobilization followed by addition of non-aqueous liquid barrier) and Group 2 (Pellet immobilization with DNA IQ™ Resin alone).

Group 1: 100 μl Differex™ Separation Solution (dimethyl glutarate) (cat#A8512) was then added to each sample tube near the bottom of the aqueous digest but above the magnetized Resin to cover the resin and cell pellet. The aqueous digest fraction and the Separation Solution separated into an upper aqueous layer and a lower non-aqueous layer. The upper aqueous layer was removed. 500 μl Nuclease-Free Water (Promega Corp., Cat#P1193) was added gently to each tube to wash tube walls and upper surface of Separation Solution. Sample tubes were returned to the swinging-bucket rotor to spin for an additional 10 minutes at 1,400×g. Following the spin, the water wash was removed and discarded. The wash step was repeated twice more for a total of three water washes. 250 μl DNA IQ™ Lysis Buffer with DTT to 60 mM was then added to samples and the samples were vortexed briefly. The resultant samples were used as starting material for DNA IQ™ nucleic acid isolation.

Group 2: Most of the aqueous epithelial digest was removed, leaving about 20 μl residual volume in sample tube above the Resin-immobilized cell pellet. 500 μl Nuclease-Free Water was gently added to sample tubes to dilute epithelial cell DNA present in the supernatant. The wash volume was removed, leaving 200 residual volume. The wash process was repeated twice, for a total of three washes. 250 μl DNA IQ™ Lysis Buffer, with DTT added to 60 mM, was added to samples and the samples were vortexed briefly. The resultant samples were used as starting material for DNA IQ™ nucleic acid isolation.

Sperm fractions from both treatment groups were transferred into wells of a 96-well plate with 2.2 ml capacity per well (ABgene catalog #AB-0932, Rochester, N.Y.). DNA was isolated from the samples in the plate using DNA IQ™ nucleic acid isolation automated on the Biomek® 2000 Workstation (Beckman Coulter). Isolated DNA was eluted from each sample in a 100 μl volume.

Each DNA preparation was quantified for male-specific DNA content using a Plexor™ (Promega catalog #A4011) quantitative PCR assay targeted to the Y-chromosome. 0.25 ng male-specific DNA from each sperm-fraction preparation was used as template for THO1 single-locus STR amplification. At the THO1 locus, both female and male contributors were heterozygous. The female contributor displayed a genotype of (9.3, 9) and the male contributor displayed a genotype of (9, 6). Both contributors shared the 9-repeat allele at the THO1 locus. The unshared THO1 alleles between of the two contributors were 9.3 repeats (female-specific) and 6 repeats (male-specific). Electropherogram peaks for the 9.3 and 6 base pair-repeat amplification products displayed in the fluorescein channel at approximately 175 and 161 base pairs, respectively.

Examination of the fluorescent strengths of these peaks, manifested as peak heights from the electropherograms, was used to determine relative representation of the two contributors in the male fraction samples. The results obtained for the two unshared allele peaks (female- and male-specific) are presented in Table 2 below.

TABLE 2

| | 161 bp (male-specific) | 175 bp (female-specific) |
|---|---|---|
| Sample | peak height in Relative | peak height in Relative |
| Sperm Fractions | Fluorescence Units | Fluorescence Units |
| Treatment | 1565 | 0 |
| Group 1 | | |
| Sample 1 | | |
| Treatment | 2061 | 133 |
| Group 1 | | |
| Sample 2 | | |
| Treatment | 1793 | 0 |
| Group 1 | | |
| Sample 3 | | |
| Treatment | 1057 | 0 |
| Group 2 | | |
| Sample 1 | | |
| Treatment | 1447 | 118 |
| Group 2 | | |
| Sample 2 | | |
| Treatment | 2101 | 0 |
| Group 2 | | |
| Sample 2 | | |

These data show that under certain conditions, immobilization of a sperm pellet using a paramagnetic resin as a barrier performs comparably in separating sperm from a minority component in a mixture with female epithelial cells both with (Group 1) or without (Group 2) addition of a non-aqueous liquid to augment the barrier.

### Example 3

### Immobilization of Cell Pellets from Lysate Following Differential Lysis of Different Genera of Cells

Gram positive Staphylococcus aureus cells from a fresh urine sample (from a healthy individual, with bacteria added) were separated from gram negative bacteria E. coli strain JM109 (pMGFP), on the basis of initial digestion of the mixture with lysozyme, which lysed E. coli but not Staphylococcus aureus. Control samples of each bacterial strain alone were run in octuplicate in parallel to the samples below. Additionally, controls which did not use any enzyme (2×8=16) and others that used both lysozyme and lysostaphin in the initial lysis step (2×8=16) were also included. None of these control samples were overlain with particles.

In Corning round bottom 96 well plates, 5 μl of 10 mg/ml lysozyme (Sigma Aldrich, St. Louis, Mo. cat #L-6876) was added per well to 180 μl of urine containing 50 mM EDTA and about 3×10⁷ S. aureus and about 1×10⁸ E. coli JM109 (pMGFP), and incubated for 30 minutes at 37° C. After the differential lysis of the E. coli by lysozyme digestion, the S. aureus cells were pelleted by centrifugation in Corning round bottom 96 well plates at 800×g for 20 minutes using a Sorvall RT6000B centrifuge (Thermo Electron, Waltham Mass.) with a swinging bucket rotor. The resulting pellets were then overlaid with either 2 μl of Promega's DNA-IQ resin (4×8=32) (100 mg per ml, catalog #DC6701), or (for the remaining samples) “no particles added” (4×8=32 samples), 2 μl of water was added instead of DNA IQ® resin. The samples were kept for 60 seconds so the added particles would settle to the bottom, covering the pellets. Then the application of magnetic force from a flat magnet placed below the plate immobilized the DNA IQ® resin particles over the S. aureus pellets while the overlaying fluid (containing the lysed E. coli) was removed. Using Promega's Wizard Genomic DNA Purification System, the following protocol was followed for all samples:
a) 8 μl of lysostaphin (2 mg/ml) (Sigma-Aldrich, St. Louis, Mo. cat #L0761) was added per well, and the pellet resuspended by pipetting (up and down) 6 times, and incubated at 37° C. for 30 minutes. Then 90 μl of Nuclei Lysis Solution (cat #A7943) was added per well and mixed by pipetting 6 times. The plate was incubated at 21° C. for 10 minutes.
b) 35 μl of Protein Precipitation Solution (cat#A7953) was added, and the samples mixed 10 times by pipette. The plate was incubated at 21° C. for 5 minutes.
c) The plate was centrifuged at 800×g for 20 minutes.
d) Each supernatant was transferred to a clean Corning round bottom 96 well plate containing 140 μl of isopropanol per well. Samples were mixed by 10 pipettings and left at 21° C. for 15 minutes. The plate was centrifuged at 800×g for 20 minutes. Supernatants were removed by pipet.
e) 200 μl of 75% ethanol was added per well.
f) The plate was centrifuged at 800×g for 20 minutes.
g) The supernatant was pipetted off and the plate was air dried for 20 minutes.
h) 50 μl of Elution Buffer, Blood (cat#MD1421) was added per well.
i) The plate was left at 4° C. for 12 hours. 12 μl of each sample was pooled into one 96 μl averaged sample per column, which (after 12,000×g for 30 seconds) was quantitated by absorbance at 260 nm. Individual samples were quantitated by Picogreen® (Invitrogen, Carlsbad, Calif.).
   The results, shown in Table 3, indicate that the pellet immobilization cap method gives higher average DNA yields.

TABLE 3

| Plate A Samples | ng/ul | Plate A | Plate B Samples | ng/ul | Plate B |
|---|---|---|---|---|---|
| column 1 no particles A | 1.25 | | Column 1 + 2 ul DNAIQ resin A | 1.76 | |
| column 1 no particles B | 1.75 | | Column 1 + 2 ul DNAIQ resin B | 1.92 | |
| column 1 no particles C | 1.59 | | Column 1 + 2 ul DNAIQ resin C | 1.65 | |
| column 1 no particles D | 1.83 | | Column 1 + 2 ul DNAIQ resin D | 2.31 | |
| column 1 no particles E | 1.86 | | Column 1 + 2 ul DNAIQ resin E | 2.18 | |
| column 1 no particles F | 2.04 | | Column 1 + 2 ul DNAIQ resin F | 1.94 | |
| column 1 no particles G | 1.79 | Average | Column 1 + 2 ul DNAIQ resin G | 2.56 | Average |
| column 1 no particles H | 2.57 | 1.83 | Column 1 + 2 ul DNAIQ resin H | 2.48 | 2.10 |
| column 2 no particles A | 1.97 | | Column 2 + 2 ul DNAIQ resin A | 2.02 | |
| column 2 no particles B | 1.85 | | Column 2 + 2 ul DNAIQ resin B | 1.88 | |
| column 2 no particles C | 1.26 | | Column 2 + 2 ul DNAIQ resin C | 1.34 | |
| column 2 no particles D | 1.53 | | Column 2 + 2 ul DNAIQ resin D | 1.82 | |
| column 2 no particles E | 2.20 | | Column 2 + 2 ul DNAIQ resin E | 1.73 | |
| column 2 no particles F | 1.78 | | Column 2 + 2 ul DNAIQ resin F | 1.78 | |
| column 2 no particles G | 1.36 | Average | Column 2 + 2 ul DNAIQ resin G | 1.61 | Average |
| column 2 no particles H | 1.99 | 1.74 | Column 2 + 2 ul DNAIQ resin H | 1.86 | 1.75 |
| column 3 + 2 ul DNA_IQ resin | 2.15 | | column 3 no particles added A | 1.36 | |
| column 3 + 2 ul DNA_IQ resin | 1.91 | | column 3 no particles added B | 1.14 | |
| column 3 + 2 ul DNA_IQ resin | 1.65 | | column 3 no particles added C | 1.42 | |
| column 3 + 2 ul DNA_IQ resin | 2.30 | | column 3 no particles added D | 1.20 | |
| column 3 + 2 ul DNA_IQ resin | 2.15 | | column 3 no particles added E | 1.14 | |
| column 3 + 2 ul DNA_IQ resin | 2.26 | | column 3 no particles added F | 1.31 | |
| column 3 + 2 ul DNA_IQ resin | 2.81 | Average | column 3 no particles added G | 1.93 | Average |
| column 3 + 2 ul DNA_IQ resin | 2.83 | 2.26 | column 3 no particles added H | 2.23 | 1.47 |
| column 4 + 2 ul DNA_IQ resin | 2.32 | | column 4 no particles added A | 1.24 | |
| column 4 + 2 ul DNA_IQ resin | 1.84 | | column 4 no particles added B | 1.34 | |
| column 4 + 2 ul DNA_IQ resin | 1.59 | | column 4 no particles added C | 0.96 | |
| column 4 + 2 ul DNA_IQ resin | 2.11 | | column 4 no particles added D | 1.13 | |
| column 4 + 2 ul DNA_IQ resin | 2.19 | | column 4 no particles added E | 0.99 | |
| column 4 + 2 ul DNA_IQ resin | 1.73 | | column 4 no particles added F | 0.96 | |
| column 4 + 2 ul DNA_IQ resin | 2.01 | Average | column 4 no particles added G | 1.39 | Average |
| column 4 + 2 ul DNA_IQ resin | 3.38 | 2.15 | column 4 no particles added H | 1.21 | 1.15 |
| column 5 no enzyme control | 1.55 | | column 5 both enzymes added A | 0.68 | |
| column 5 no enzyme control | 1.08 | | column 5 both enzymes added B | 0.60 | |
| column 5 no enzyme control | 1.09 | | column 5 both enzymes added C | 0.62 | |
| column 5 no enzyme control | 0.94 | | column 5 both enzymes added D | 0.63 | |
| column 5 no enzyme control | 0.91 | | column 5 both enzymes added E | 0.76 | |
| column 5 no enzyme control | 0.68 | | column 5 both enzymes added F | 0.58 | |
| column 5 no enzyme control | 1.04 | Average | column 5 both enzymes added G | 0.73 | Average |
| column 5 no enzyme control | 1.12 | 1.05 | column 5 both enzymes added H | 0.75 | 0.67 |
| column 6 no enzyme control | 0.71 | | column 6 both enzymes added A | 0.91 | |
| column 6 no enzyme control | 0.62 | | column 6 both enzymes added B | 0.65 | |
| column 6 no enzyme control | 0.76 | | column 6 both enzymes added C | 0.68 | |
| column 6 no enzyme control | 0.77 | | column 6 both enzymes added D | 0.80 | |
| column 6 no enzyme control | 1.40 | | column 6 both enzymes added E | 0.57 | |
| column 6 no enzyme control | 0.87 | | column 6 both enzymes added F | 0.45 | |
| column 6 no enzyme control | 1.55 | Average | column 6 both enzymes added G | 0.33 | Average |
| column 6 no enzyme control | 1.90 | 1.07 | column 6 both enzymes added H | 0.28 | 0.58 |
| column 7 JM109 (pMGFP) | 0.80 | | col 7 S. aureus no lysostaphin A | 0.16 | |
| column 7 JM109 (pMGFP) | 0.83 | | col 7 S. aureus no lysostaphin B | 0.18 | |
| column 7 JM109 (pMGFP) | 0.80 | | col 7 S. aureus no lysostaphin C | 0.16 | |
| column 7 JM109 (pMGFP) | 0.95 | | col 7 S. aureus no lysostaphin D | 0.16 | |
| column 7 JM109 (pMGFP) | 0.28 | | col 7 S. aureus + lysostaphin A | 0.39 | |
| column 7 JM109 (pMGFP) | 0.34 | | col 7 S. aureus + lysostaphin B | 0.32 | |
| column 7 JM109 (pMGFP) | 0.54 | Average | col 7 S. aureus + lysostaphin C | 0.21 | Average |
| column 7 JM109 (pMGFP) | 0.80 | 0.67 | col 7 S. aureus + lysostaphin D | 0.33 | 0.24 |

### Example 4

### Isolation of DNA from Immobilized Pelleted Nuclei

A 50 ul aliquot of human whole blood was diluted in 150 ul of Red Cell Lysis buffer, using the Wizard® Genomic DNA Purification System (Promega cat number A1120). Red blood cells and white blood cells were lysed, while the nuclei of white blood cells remained intact. The nuclei were pelleted by centrifugation at 1400×g in a Corning round bottom 96 well plate for 10 minutes using a swinging bucket rotor. In one half of the samples, the resulting pellets were overlaid with 20 ul of Promega's MagneSil® paramagnetic particles (catalog #A2201) to cover the pelleted nuclei with a layer of magnetic particles. The other half of the samples were contacted with 20 ul of water was added instead of MagneSil® particles. An application of magnetic force was applied by placing a magnet placed below the plate to immobilize the pelleted nuclei while the overlaying supernatant was removed.

### Example 5

### Isolation of DNA from Immobilized Pelleted Nuclei

DNA was isolated from each sample using the Wizard Genomic DNA Purification System following the standard protocol. Briefly, 100 ul of Nuclei Lysis Solution (Promega cat #A7943) was added per well and mixed by pipetting 6 times. The plate was incubated at 21° C. for 10 minutes. 35 ul of Protein Precipitation Solution (cat#A7953) was added, and the samples mixed 6 times by pipet. The plate was incubated at 21° C. for 5 minutes. The plate was centrifuged at 800×g for 20 minutes. Each supernatant was transferred to a clean Corning round bottom 96 well plate containing 140 ul of isopropanol per well. The plate was centrifuged at 800×g for 20 minutes. Supernatants were removed by pipet. 200 ul of 75% ethanol was added per well. The plate was centrifuged at 800×g for 20 minutes. The supernatant was pipetted off and the plate was air dried for 30 minutes. 50 ul of Elution Buffer, Blood (cat#MD1421) was added per well. The plate was incubated at 4° C. for 12 hours. 12 ul of each sample was pooled into one 96 ul averaged sample per column, which (after 12,000×g for 30 seconds) was quantitated by absorbance at 260 nm (minus A320). Individual samples were quantitated by Picogreen® (Invitrogen, Carlsbad, Calif.). The results are shown in Tables 4 and 5.

TABLE 4

| Plate 1 Samples | ng/ul | | Plate 2 Samples | ng/ul | |
|---|---|---|---|---|---|
| Column 1 “0 ul” | 6.6 | | Column 1 “2 ul” | 8.4 | |
| Column 1 “0 ul” | 3.8 | | Column 1 “2 ul” | 12.2 | |
| Column 1 “0 ul” | 2.5 | | Column 1 “2 ul” | 6.5 | |
| Column 1 “0 ul” | 0.0 | | Column 1 “2 ul” | 3.6 | |
| Column 1 “0 ul” | 0.0 | | Column 1 “2 ul” | 13.0 | |
| Column 1 “0 ul” | 0.0 | | Column 1 “2 ul” | 13.1 | |
| Column 1 “0 ul” | 0.4 | Avg | Column 1 “2 ul” | 17.6 | Avg |
| Column 1 “0 ul” | 3.7 | 2.1 | Column 1 “2 ul” | 19.3 | 11.7 |
| Column 2 “0 ul” | 10.4 | | Column 2 “2 ul” | 0.0 | |
| Column 2 “0 ul” | 4.2 | | Column 2 “2 ul” | 0.6 | |
| Column 2 “0 ul” | 1.1 | | Column 2 “2 ul” | 8.2 | |
| Column 2 “0 ul” | 10.9 | | Column 2 “2 ul” | 7.6 | |
| Column 2 “0 ul” | 0.8 | | Column 2 “2 ul” | 11.4 | |
| Column 2 “0 ul” | 0.9 | | Column 2 “2 ul” | 19.5 | |
| Column 2 “0 ul” | 1.5 | Avg | Column 2 “2 ul” | 16.1 | Avg |
| Column 2 “0 ul” | 3.3 | 4.1 | Column 2 “2 ul” | 11.7 | 9.4 |
| Column 3 “4 ul” | 1.6 | | Column 3 “1 ul” | 12.7 | |
| Column 3 “4 ul” | 4.1 | | Column 3 “1 ul” | 16.4 | |
| Column 3 “4 ul” | 3.2 | | Column 3 “1 ul” | 12.1 | |
| Column 3 “4 ul” | 9.0 | | Column 3 “1 ul” | 4.5 | |
| Column 3 “4 ul” | 7.3 | | Column 3 “1 ul” | 0.0 | |
| Column 3 “4 ul” | 11.2 | | Column 3 “1 ul” | 8.6 | |
| Column 3 “4 ul” | 13.9 | Avg | Column 3 “1 ul” | 4.5 | Avg |
| Column 3 “4 ul” | 10.2 | 7.6 | Column 3 “1 ul” | 0.6 | 7.4 |
| Column 4 “4 ul” | 0.0 | | Column 4 “1 ul” | 7.9 | |
| Column 4 “4 ul” | 0.8 | | Column 4 “1 ul” | 10.7 | |
| Column 4 “4 ul” | 7.2 | | Column 4 “1 ul” | 9.2 | |
| Column 4 “4 ul” | 9.1 | | Column 4 “1 ul” | 6.7 | |
| Column 4 “4 ul” | 3.4 | | Column 4 “1 ul” | 6.9 | |
| Column 4 “4 ul” | 8.8 | | Column 4 “1 ul” | 16.0 | |
| Column 4 “4 ul” | 11.8 | Avg | Column 4 “1 ul” | 14.6 | Avg |
| Column 4 “4 ul” | 15.4 | 7.1 | Column 4 “1 ul” | 13.2 | 10.7 |
| Column 5 “0 ul” | 6.2 | | Column 5 “1 ul” | 25.6 | |
| Column 5 “0 ul” | 6.0 | | Column 5 “1 ul” | 13.8 | |
| Column 5 “0 ul” | 8.5 | | Column 5 “1 ul” | 11.6 | |
| Column 5 “0 ul” | 8.2 | | Column 5 “1 ul” | 7.0 | |
| Column 5 “0 ul” | 9.3 | | Column 5 “1 ul” | 9.5 | |
| Column 5 “0 ul” | 3.3 | | Column 5 “1 ul” | 17.8 | |
| Column 5 “0 ul” | 6.3 | Avg | Column 5 “1 ul” | 10.6 | Avg |
| Column 5 “0 ul” | 9.1 | 7.1 | Column 5 “1 ul” | 7.8 | 13.0 |
| Column 6 “0 ul” | 2.1 | | Column 6 “2 ul” | 3.3 | |
| Column 6 “0 ul” | 1.0 | | Column 6 “2 ul” | 3.5 | |
| Column 6 “0 ul” | 2.4 | | Column 6 “2 ul” | 3.7 | |
| Column 6 “0 ul” | 2.3 | | Column 6 “2 ul” | 3.0 | |
| Column 6 “0 ul” | 3.7 | | Column 6 “2 ul” | 5.3 | |
| Column 6 “0 ul” | 3.1 | | Column 6 “2 ul” | 7.3 | |
| Column 6 “0 ul” | 3.3 | Avg | Column 6 “2 ul” | 12.7 | Avg |
| Column 6 “0 ul” | 2.0 | 2.5 | Column 6 “2 ul” | 15.5pan> | 6.8 |

TABLE 5

| | | | | | | picogreen | | ng/ul pg |
|---|---|---|---|---|---|---|---|---|
| | | | | A260 | 50 ul | average per | picogreen | class |
| sample | A260 | A280 | A320 | ng/ul | yield ug | column | ug yield | average |
| none A | 0.0316 | 0.0133 | 0 | 1.58 | 0.079 | 2.1 | 0.105 | 3.95 |
| none B | 0.1373 | 0.067 | 0 | 6.865 | 0.34325 | 4.1 | 0.205 | |
| none C | 0.1426 | 0.0705 | 0 | 7.13 | 0.3565 | 7.1 | 0.355 | |
| none D | 0.0613 | 0.0295 | 0 | 3.065 | 0.15325 | 2.5 | 0.125 | |
| 1 ul A | 0.1577 | 0.0778 | 0 | 7.885 | 0.39425 | 7.2 | 0.36 | 10.1 |
| 1 ul B | 0.2102 | 0.1052 | 0 | 10.51 | 0.5255 | 10.4 | 0.52 | |
| 1 ul C | 0.2934 | 0.1471 | 0 | 14.67 | 0.7335 | 12.7 | 0.635 | |
| 2 ul A | 0.2484 | 0.1256 | 0 | 12.42 | 0.621 | 11.5 | 0.575 | 9.1 |
| 2 ul B | 0.2003 | 0.0995 | 0 | 10.015 | 0.50075 | 9.2 | 0.46 | |
| 2 ul C | 0.1357 | 0.0663 | 0 | 6.785 | 0.33925 | 6.6 | 0.33 | |
| 4 ul A | 0.1493 | 0.0737 | 0 | 7.465 | 0.37325 | 7.6 | 0.38 | 7.35 |
| 4 ul B | 0.1373 | 0.067 | 0 | 6.865 | 0.34325 | 7.1 | 0.355 | |

As can be seen in Table 4 (averaged values for each column (group of eight samples) per sample category) and Table 5 (picogreen values for individual samples, and averages thereof), the use of 1 ul, 2 ul or 4 ul of MagneSil magnetic particles (1 ug, 2 ug or 4 ug of particles per well) provided higher average yields than did samples without MagneSil® particle overlay. Additionally, the Picogreen results indicate that there was a lower frequency of sample dropouts (i.e., samples in which no DNA was detected) when the nuclei pellets were immobilized using MagneSil® particles. The use of magnetic particles to immobilize the pelleted nuclei provides for more uniformly reliable results and higher DNA yields. Presumably, fewer cell nuclei in the pellet were lost per sample, and fewer nuclei pellets in aggregate were lost when the supernatants were removed from samples in which magnetic particles were used to immobilize the pellets.

### Example 6

### Purification of Cytoplasmic RNA Away from Nuclei

Cytoplasmic RNA is isolated from each sample using 200 ul per well in a 96 well plate of a lysis solution comprising 100 mM HEPES pH 7.6/100 mM EDTA/40 mg/ml digitonin/0.4% DMSO (vol/vol)/400 units of RNasin Plus (Promega catalog N2611). Briefly, 200 ul of Lysis Solution containing 1×10⁶ HEK 293 cells is added per well and mixed by pipetting 6 times. The plate is incubated at 4° C. for 10 minutes. The plate is centrifuged at 800×g for 20 minutes. Every other sample pellet (e.g. in a 96 well plate wells A1, A3, A5, A7, B2, B4, B6, etc) is overlaid with 1 ul of MagneSil Blue particles, and the remaining sample pellets are not overlaid with MagneSil Blue particles. The plate is then placed on a flat magnet for 60 seconds. The supernatants are then transferred to their corresponding wells by use of a multi-channel pipetter into a clean Corning round bottom 96 well plate. Quantitation of DNA per sample well is obtained using Promega's DNA Quantitation system (catalog number K4000), and quantitation of RNA is obtained using Quant-iT RiboGreen (Invitrogen catalog R11490, Carlsbad, Calif.). The RNA is run in quantitative RT-PCR using Promega's Plexor 2-step qRT-PCR System (catalog number A4051).

### Example 7

### Purification of Human Sperm Away from Epithelial Cell Debris

Human sperm is isolated from each sample using 200 ul per well in a 96 well plate of a lysis solution comprising 100 mM HEPES pH 7.6/100 mM EDTA/40 mg/ml digitonin/0.4% DMSO (vol/vol). Briefly, 200 ul of Lysis Solution containing 1×10³ human sperm cells and 1×10⁶ human epithelial cells is added per well and mixed by pipetting 6 times. The plate is incubated at 4° C. for 10 minutes. The plate is centrifuged at 800×g for 20 minutes. Every other sample pellet (e.g. in a 96 well plate wells A1, A3, A5, A7, B2, B4, B6, etc) is overlaid with 1 ul of MagneSil Blue particles, and the remaining sample pellets are not overlaid with MagneSil Blue particles. The plate is then placed on a flat magnet for 60 seconds. The supernatants are then transferred to their corresponding wells by use of a multi-channel pipetter into a clean Corning round bottom 96 well plate. The sperm pellet is purified using Promega's Differex System (catalog number DC6800), and the DNA obtained is quantitated using Promega's AluQuant Human DNA Quantitation System (catalog number DC1010), and profiled using Promega's PowerPlex 16 System (catalog number DC6530).

1. A method of isolating first cells from a sample comprising first cells and second cells comprising:
(a) treating the sample to form an aqueous lysate under conditions that lyse the second cells without lysing the first cells;
(b) applying a force to the sample for a period of time sufficient to form a pellet comprising the first cells; and
(c) forming a pellet immobilizing cap between the pellet and the aqueous lysate.

2. The method of claim 1, wherein the first cells are sperm cells.

3. The method of claim 1, further comprising removing the aqueous lysate of step (c).

4. The method of claim 1, further comprising recovering the pellet.

5. The method of claim 4, further comprising washing the pellet.

6. The method of claim 3, further comprising, prior to removing the aqueous lysate, contacting the aqueous lysate of step (c) with a non-aqueous liquid having a density of greater than about 1.00 g/cm³ to form a non-aqueous layer between the pellet immobilizing cap and the aqueous lysate.

7. The method of claim 1, wherein the force is applied by centrifugation.

8. The method of claim 1, wherein the pellet immobilizing cap comprises magnetic or paramagnetic material.

9. The method of claim 8, further comprising applying a magnetic field to the magnetic or paramagnetic material to maintain the cap between the sperm pellet and aqueous material.

10. The method of claim 8, wherein the paramagnetic material comprises silica.

11. The method of claim 8, wherein the paramagnetic material comprises silica magnetic particles.

12. The method of claim 8, wherein the paramagnetic material comprises siliceous oxide coated magnetic particles.

13. The method of claim 8, wherein the paramagnetic material comprises an aggregate magnetic particle wherein each particle comprises two or more magnetic or paramagnetic cores that are covered by a silaceous oxide.

14. The method of claim 8, wherein the paramagnetic material comprises a magnetic mat.

15. The method of claim 1, wherein the pellet immobilizing cap material comprises cellulosic magnetic particles.

16. The method of claim 1, wherein the pellet immobilizing cap material comprises a temperature-responsive material.

17. The method of claim 1, wherein the method is performed in a high throughput process.

18. The method of claim 7, wherein the force is about 3000×g or less.

19. The method of claim 1, wherein the second cells are epithelial cells.

20. The method of claim 1, wherein the aqueous material comprises non-sperm cell DNA.

21. The method of claim 6, wherein the non-aqueous liquid comprises at least one of diethyl glutarate, dimethyl glutarate, and 1-chloro-2-methyl-2-propanol.

22. The method of claim 21, wherein the non-aqueous liquid further comprises chloroform.

23. The method of claim 22, wherein the non-aqueous liquid comprises chloroform and dimethyl glutarate in a ratio of from about of 0.1:99.9 to about 50:50 dimethyl glutarate:chloroform.

24. The method of claim 10, further comprising lysing the first cells under conditions that allow binding of first cell DNA to the silica.

25. The method of claim 15, further comprising lysing the first cells under conditions that allow binding of the DNA from the first cells to the cellulosic material.

26. The method of claim 1, wherein the aqueous lysate comprises digitonin in a concentration effective to lyse the second cells without lysing the first cells.

27. A method of isolating sperm cell DNA from an aqueous sample comprising epithelial cells comprising:
(a) treating the sample under conditions that allow selective lysis of the epithelial cells;
(b) applying a force to the treated sample for a period of time sufficient to form a sperm pellet;
(c) forming a pellet immobilizing cap between the sperm pellet and the aqueous material containing lysed epithelial cells;
(d) removing the aqueous material; and
(e) lysing the cells in the sperm pellet under conditions that allow sperm cell DNA purification.

28. The method of claim 27, further comprising, prior to step (d), adding a non-aqueous liquid having a density of greater than about 1.00 g/cm³ to form a non-aqueous layer between the pellet immobilizing cap and the lysed epithelial cells.

29. The method of claim 28, further comprising:
(f) after step (d) and before step (e), washing the non-aqueous liquid, the pellet immobilizing cap, and the sperm cells;
(g) applying a force to form a wash layer, a non-aqueous layer, and a pellet comprising the pellet immobilizing cap material and sperm cells;
(h) after step (g), adding a paramagnetic material comprising silica to form a paramagnetic pellet immobilizing cap between the pellet of step (g) and the non-aqueous layer; and
(i) removing the wash layer and the non-aqueous layer.

30. A method of separating intact cells or target organelles from an aqueous cell lysate sample comprising:
(a) applying a force to the sample for a period of time sufficient to form a pellet comprising at least some of the intact cells or target organelles; and
(b) forming a pellet immobilizing cap between the pellet and the aqueous cell lysate.

31. The method of claim 30, further comprising removing the aqueous material.

32. The method of claim 30, further comprising recovering the pellet.

33. The method of claim 30, wherein the force is applied by centrifugation.

34. The method of claim 30, wherein the pellet immobilizing cap comprises magnetic or paramagnetic material.

35. The method of claim 34, further comprising applying a magnetic field to the paramagnetic material to maintain the pellet immobilizing cap between the pellet and aqueous material.

36. A method of isolating target organelles from a sample comprising cells comprising a target organelle comprising:
(a) treating the sample to form an aqueous lysate under conditions that selectively lyse the cells without lysing the organelles;
(b) applying a force to the sample for a period of time sufficient to form a pellet comprising the target organelles; and
(c) forming a pellet immobilizing cap between the pellet and the lysate.

37. The method of claim 36, wherein treating the sample of step (a) comprises contacting the sample with a lysis buffer comprising digitonin in a concentration effective to cause the selective lysis of step (a).

38. The method of claim 37, wherein the lysis buffer further comprises RNasin.

39. A method of isolating a target cell substituent from a sample comprising target cells comprising:
(a) treating the sample to form an aqueous lysate under conditions that selectively lyse the target cells and under which at least one organelle is not lysed;
(b) applying a force to the sample for a period of time sufficient to form a pellet comprising the unlysed organelles; and
(c) forming a pellet immobilizing cap between the pellet and the lysate.

40. The method of claim 39, wherein the pellet comprises nuclei.

41. The method of claim 39, wherein treating the sample of step (a) comprises contacting the sample with a lysis buffer comprising digitonin in a concentration effective to cause the selective lysis of step (a).

42. The method of claim 41, wherein the lysis buffer further comprises RNasin.

43. A kit for isolating a target cell or organelle from a non-target cell comprising a lysis agent for preferentially lysing the non-target cell and a pellet immobilizing material.

44. The kit of claim 32, wherein the pellet immobilizing material comprises at least one of a silica magnetic particle, a cellulosic magnetic material, a temperature-responsive material, and a magnetic mat.
